(19) United States
(12) Patent Application Publication
(10) Pub. No.: US 2025/0352599 A1
(43) Pub. Date: Nov. 20, 2025

(54) METHODS OF TREATING TOPICAL CONDITIONS

(71) Applicant: ANSWERS BY NATURE, North Hollywood, CA (US)

(72) Inventors: Mote London, North Hollywood, CA (US); Cheryl Smith, North Hollywood, CA (US)

(21) Appl. No.: 19/207,903

(22) Filed: May 14, 2025

Related U.S. Application Data
(60) Provisional application No. 63/648,088, filed on May 15, 2024.

(51) Int. Cl. A61K 36/66; A61K 9/00; A61K 36/48; A61K 36/70; A61K 36/9068; A61P 17/02

(52) U.S. Cl. CPC A61K 36/66; A61K 9/0014; A61K 36/48; A61K 36/70; A61K 36/9068; A61P 17/02

(57) ABSTRACT

A method of treating a subject with a topical condition, comprising applying to a topical surface of the subject a composition comprising red clover, bloodroot, galangal, and sheep sorrel, wherein the topical condition is an infection, animal bite, or dermatitis, and wherein the composition is kept on the topical surface for no more than 12 hours without a cover.

### CROSS REFERENCE TO RELATED APPLICATION

This application claims the benefit of, and priority to, U.S. Provisional Application No. 63/648,088, filed May 15, 2024. The entire contents of the above-identified application are hereby fully incorporated herein by reference.

### TECHNICAL FIELD

The subject matter disclosed herein is generally directed to novel methods of treating topical conditions with a composition comprising natural ingredients.

### BACKGROUND

The treatment of topical conditions such as infection, animal bites, and dermatitis faces challenges such as suboptimal efficacy and resistance to conventional antibiotics and other topical treatment medicines. There is a need for novel and alternative treatments for these conditions.

### SUMMARY

The present disclosure provides novel methods of treating topical conditions. In one aspect, the present disclosure provides a method of treating a subject with a topical condition, comprising applying to the topical surface of the subject a composition comprising red clover, bloodroot, galangal, and sheep sorrel, wherein the topical condition is an infection, animal bite, or dermatitis, and wherein the composition is kept on the topical surface for no more than 12 hours without a cover.

In some embodiments, the topical surface is the skin. In some embodiments, the topical surface is oral mucosa.

In some embodiments, the composition is kept on the topical surface for no more than 1 hour. In some embodiments, the composition is kept on the topical surface for no more than 5 minutes. In some embodiments, the composition is kept on the topical surface for no more than 2 minutes. In some embodiments, the composition is kept on the topical surface for no more than 1 minute.

In some embodiments, the topical condition is an infection. In some embodiments, the infection is a bacterial or fungal infection. In some embodiments, the infection is cellulitis, staph, MRSA, or jock itch. In some embodiments, the topical condition is an animal bite. In some embodiments, the topical condition is an insect bite or spider bite. In some embodiments, the topical condition is dermatitis. In some embodiments, the dermatitis is eczema, psoriasis, or rash.

In some embodiments, the composition is a paste, ointment, or salve. In some embodiments, the method further comprises applying the composition to the topical surface more than one time.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention may be utilized, and the accompanying drawings of which:

FIGS. 1A-1I show the treatment of an antibiotic-resistant infection with an exemplary salve according to the present disclosure. FIG. 1A shows an infection area after antibiotic treatments. FIG. 1B shows another view of the infection area of FIG. 1A. FIG. 1C shows the area after treated by an exemplary salve. FIG. 1D shows the treated area a few hours after the salve application. FIG. 1E shows the treated area one day after the salve application. FIG. 1F shows the treated area 2 days after the salve application. FIG. 1G shows the treated area 5 days after the salve application. FIG. 1H shows the treated area 10 days the salve application. FIG. 1I shows the treated area a few months after the treatment.

FIGS. 2A-2D show the treatment of an infected spider bite with an exemplary salve according to the present disclosure. FIG. 2A shows infected area. FIG. 2B shows the area after the salve application. FIG. 2C shows the treated area 24 hours after the salve application. FIG. 2D shows the area 5 days after the salve application.

### DETAILED DESCRIPTION OF THE EXAMPLE EMBODIMENTS

### General Definitions

As used herein, the singular forms “a”, “an”, and “the” include both singular and plural referents unless the context clearly dictates otherwise.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The term “extract” refers to a composition prepared from a raw material (e.g., a raw material of natural source). An extract may be prepared by processing any raw materials to extract and concentrate materials and compounds contained within the raw material.

The term “ointment” refers to a semi-solid preparation including an ointment base having one or more active ingredients incorporated or fused (e.g., melted together with other components of the formulation and cooled with constant stirring to form a congealed preparation) therein. The ointment base may be in the form of: an oleaginous or hydrocarbon base (e.g., petrolatum or a petrolatum/wax combination); an absorption base that permits the incorporation of aqueous solution resulting in the formation of a water-in-oil emulsion (e.g., hydrophilic petrolatum) or that is a water-in-oil that permits the incorporation of additional quantities of aqueous solutions (e.g., lanolin); a water-removable base that is an oil-in-water emulsion that may be diluted with water or aqueous solutions (e.g., hydrophilic ointment, USP); or a water-soluble base that contains oleaginous components (e.g., polyethylene glycol (“PEG”) formulations that combine PEGs having an average molecular weight below 600 with a PEG having an average molecular weight above 1,000); and the like.

The term “paste” refers to a preparation containing a larger proportion of solid material rendering them stiffer than ointments.

The term “cream” refers to a semi-solid preparation containing one or more active or medicinal agent dissolved or dispersed in either a water-in-oil emulsion or an oil-in-water emulsion or in another type of water-washable base.

The term “gel” refers to a semi-solid system including a dispersion of small and/or large molecules in an aqueous liquid vehicle that is rendered jellylike by the addition of a gelling agent. Suitable gelling agents include, but not are limited to, synthetic macromolecules (e.g., carbomer polymers), cellulose derivatives (e.g., carboxymethylcellulose and/or hydroxypropyl methylcellulose), and natural gums (e.g., tragacanth gum, carrageenan, and the like). Gel preparations may be in the form of a single-phase gel in which the active or medicinal ingredients are uniformly dispersed throughout the liquid vehicle without visible boundaries or a two-phase gel, wherein flocculants or small distinct particles of the active or medicinal ingredient are dispersed within the liquid vehicle.

The term “salve” refers to a composition comprising materials (such as natural materials or extract thereof) dissolved in, or mixed with, a base. In some embodiments, the base comprises ethanol, sugar alcohols (e.g., mannitol, sorbitol, xylitol, lactitol, isomalt, maltitol and hydrogenated starch hydrolysates), alpha hydroxy acids (e.g., glycolic acid, lactic acid, citric acid, and mandelic acid), vinegar, propylene glycol, diethylene glycol, butylene glycol, ceramides, hyaluronic acid, urea, glycerol made from any source (e.g., a vegetable glycerin, a food grade vegetable glycerin or a non-GMO glycerin), corn syrup, maple syrup, honey, vegetable oil (e.g., canola oil, palm oil, coconut oil, soybean oil, rapeseed oil, olive oil), water, or any combination thereof.

The term “topical surface” refers to the surface of the skin or mucosal tissues. A topical surface may be the skin, or oral mucosal tissue, such as mucous membrane lining the inside of the mouth, including the tongue and gums.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

In general, the present disclosure provides for methods of treating subjects with topical conditions such as infection, animal bite, or dermatitis with a composition comprising natural ingredients. In some aspects, the methods can provide simple, fast, and effective treatment of topical conditions in daily life. In some embodiment, the methods comprise applying to the topical surface of a subject a composition comprising red clover, bloodroot, galangal, and sheep sorrel, and keeping the composition on the topical surface for no more than 12 hours without a cover. In some aspects, the methods herein provide a new use of the composition that is different from using the composition as a natural remedy for treating cancerous growths on the skin, or removing moles etc. In some embodiments, the method herein does not require keeping the composition on the skin for a prolonged time (e.g., 24 hours or longer), or keeping the treated area covered (e.g., by a bandage with Vaseline).

The composition herein may comprise a mixture of red clover, bloodroot, galangal, and sheep sorrel. Red clover may be the plant Trifolium pratense, or may be any part(s) of the plant or an extract thereof. Bloodroot may be the plant Sanguinaria canadensis, or may be any part(s) of the plant or an extract thereof. Galangal may be Alpinia galanga, Alpinia officinarum, Boesenbergia rotunda, or Kaempferia galanga, or may be any part(s) of the plant or an extract thereof. In some examples, the galangal is Alpinia galanga, or may be any part(s) of the plant or an extract thereof. Sheep sorrel may be Rumex acetosella, or may be any part(s) of the plant or an extract thereof.

In some embodiments, the composition is in a semi-solid formulation. For example, the composition may be a cream, gel, ointment, paste, or salve. In one example, the composition is an ointment. In another example, the composition is a paste. In another example, the composition is a salve.

In some embodiments, the red clover, bloodroot, galangal, and sheep sorrel in the composition have a weight ratio of 1:1:1:1, 1.5:1:1:1, 1:1.5:1:1, 1:1:1.5:1, 1:1:1:1.5, 2:1:1:1, 1:2:1:1:, 1:1:2:1, or 1:1:1:2. In one example, the red clover, bloodroot, galangal, and sheep sorrel in the composition have a weight ratio of 1:1:1:1.

The methods may be used for treating various topical conditions. In some embodiments, topical surface area on which the composition is applied is kept open and not covered, e.g., not covered by a bandage. In some embodiments, the treated area is allowed to dry and form scabs or eschars, which are allowed to slough off naturally, and the treatment facilitates the growth of new, healthy tissue and wound healing.

In some embodiments, the topical condition is an infection. In some embodiments, the topical condition is an animal bite. In some embodiments, the topical condition is dermatitis.

Examples of infection include cellulitis, staph infection, methicillin-resistant Staphylococcus aureus (MRSA) infection, or jock itch, as well as bacterial infections including staphylococcal diseases, staphylococcal scalded skin syndrome, erysipelas, folliculitis, furuncles, carbuncles, paronychial infections, and erythrasma; and fungal infections including dermatophyte infections, tinea corporis, tinea pedis, tinea unguium, tinea capitis, tinea cruris, tinea versicolor, tinea barbae, athlete's foot, yeast infections including candidiasis, such as Candida Albicans, oral candida (thrush), candidal paronychia. In one example, the topical condition is cellulitis. In another example, the topical condition is staph infection. In another example, the topical condition is MRSA infection. In another example, the topical condition is jock itch.

Examples of animal bites include insect bites (e.g., mosquito bites, bee bites, wasp bites), spider bites (e.g., black widow spider bites), stings, as well sea bather's eruption, seaweed dermatitis, swimmers itch, scombroid fish poisoning, scabies, popular urticaria, and cutaneous larva migrans. In one example, the topical condition is insect bite. In another example, the topical condition is spider bite.

The term “dermatitis” refers to a condition involving the inflammation of the skin. Examples of dermatitis include eczema, psoriasis, rash, atopic dermatitis, seborrheic dermatitis, nummular dermatitis, chronic dermatitis of hands and feet, generalized exfoliative dermatitis, stasis dermatitis, and lichen simplex chronicus. In one example, the topical condition is eczema. In another example, the topical condition is psoriasis. In another example, the topical condition is rash.

In some embodiments, after applying to the topical surface, the composition is kept on the topical surface for no more than 12 hours, no more than 6 hours, no more than 4 hours, no more than 2 hours, no more than 1 hour, no more than 30 minutes, no more than 20 minutes, no more than 10 minutes, no more than 5 minutes, no more than 3 minutes, no more than 2 minutes, no more than 1 minute, or no more than 30 second. In one example, the composition is kept on the topical surface for no more than 1 hour. In another example, the composition is kept on the topical surface for no more than 5 minutes. In another example, the composition is kept on the topical surface for no more than 2 minutes. In another example, the composition is kept on the topical surface for no more than 1 minute. Then the composition may be washed off the topical surface, e.g., with water. In some embodiments, for certain infections, e.g., deeper or more serious infections, the composition is kept on the topical surface until it dries uncovered.

For a topical condition, the treatment with the composition may be repeated one or more times. The interval between two treatments may be from one or several hours, one or several days, or one or several weeks. For example, the interval between two treatments may be 30 minutes to 1 hour, 1 hour to 3 hours, 3 hours to 6 hours, 6 hours to 12 hours, 1 day to 3 days, 3 days to 6 days, 1 week to 2 weeks, 2 weeks to 4 weeks, about 1 day, about 2 days, about 3 days, about 4 days, about 5 days, about 6 days, about 1 week, about 2 weeks, about 3 weeks, or longer. The treatment may be applied to a subarea of the topical surface and repeated on the same or different subarea(s) of the topical surface. The frequency, interval, and/or area of the treatment may depend on the reaction of the patient.

In another aspect, the present disclosure provides a method of treating a subject with a topical condition, comprising applying to a topical surface of the subject a composition comprising an extract from bloodroot. In some embodiments, the extract from bloodroot is bloodroot juice. For example, the bloodroot juice may be extracted by scrapping and/or squeezing the raw tissue (e.g., the root) of bloodroot. The bloodroot extract (e.g., bloodroot juice) may be applied by rubbing into a topical surface for treatment. In embodiments, the bloodroot extract is left on the topical surface for 5 minutes to 30 minutes, 30 minutes to 1 hour, 1 hour to 24 hours, 6 hours to 24 hours, 8 hours to 24 hours, 10 hours to 24 hours, 12 hours to 24 hours, 16 hours to 24 hours, 20 hours to 24 hours, 1 hour to 4 hours, 4 hours to 8 hours, 8 hours to 12 hours, 12 hours to 16 hours, 16 hours to 20 hours, or more than 24 hours. In some embodiments, the topical condition to be treated by the extract from bloodroot is an infection, animal bite, or dermatitis.

### EXAMPLES

### Example 1—Treatments of Various Skin Conditions

This example describes treating various skin conditions with an exemplary salve comprising red clover, bloodroot, galangal, and sheep sorrel according to the present disclosure.

The first condition was an extremely severe case of MRSA infection that had lasted for years. The patient had large sores with biofilms covering his legs and causing major health issues. The salve was applied to the skin area, and washed off after several hours. The sores were allowed to dry up and slough off. When repeated, the salve was also applied to secondary infections arising after the main sites were treated. The infection was completely healed and never returned.

Another condition was a severe black widow bite, which formed a large, lime green colored, oozing volcano-looking wound on the leg of the patient. A different formula was used to treat the condition but only reduced the condition to manageable and the wound became infected. The exemplary salve according to the present disclosure was then applied to the infected area and left for a minute before washing off. The wound scabbed and healed completely in the following weeks.

Another condition was patches of eczema over the body of a patient. The exemplary salve was applied to some of the patches for a minute and then washed off. Some of the eczema patches disappeared after a few days. The salve was applied again to the remaining patches and repeated the treatment a few times over about two weeks. The eczema patches eventually disappeared.

Another condition was a case of jock itch on a groin area. The exemplary salve was applied briefly for 15 to 30 seconds daily until the infection healed.

Another condition was a mosquito bite. The exemplary salve was applied to the area of bite for about a minute. The treatment stopped itching and healed the bite quickly.

### Example 2

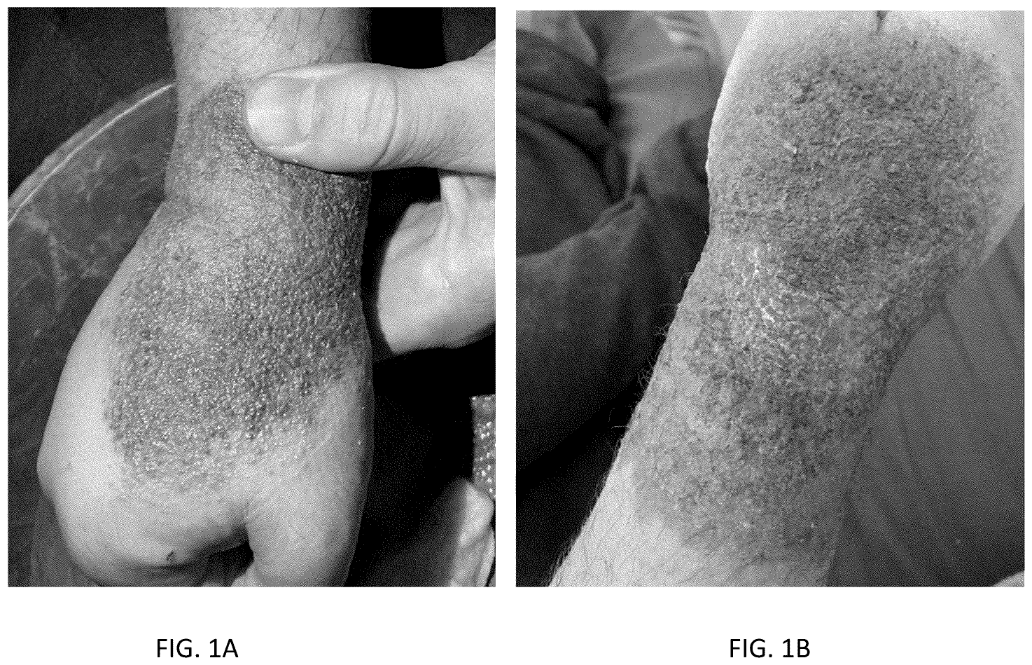

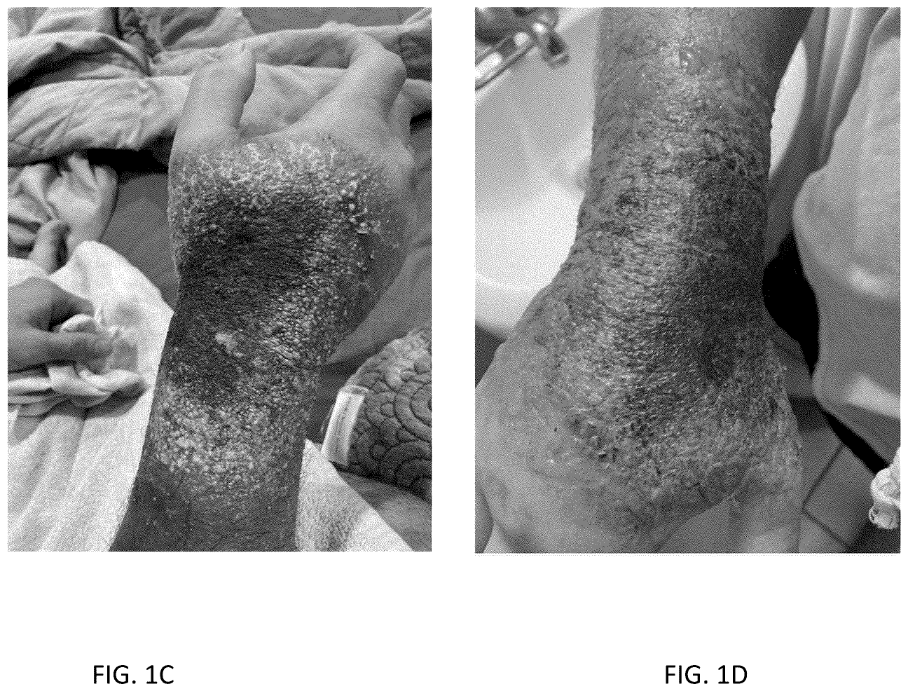

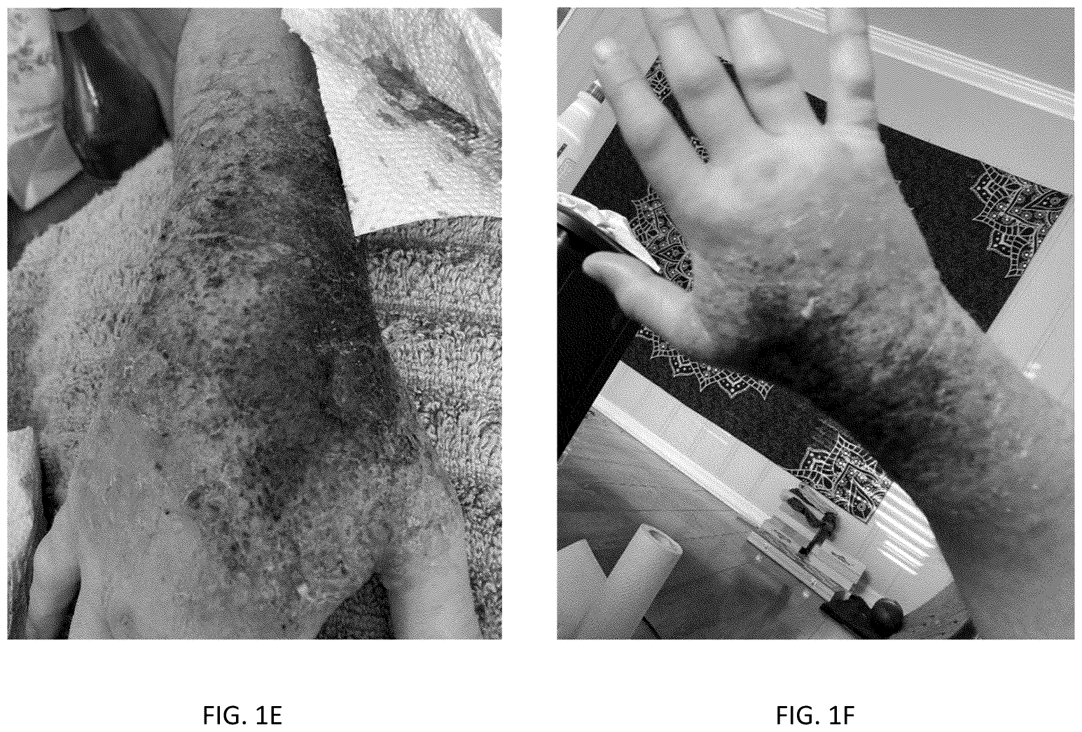

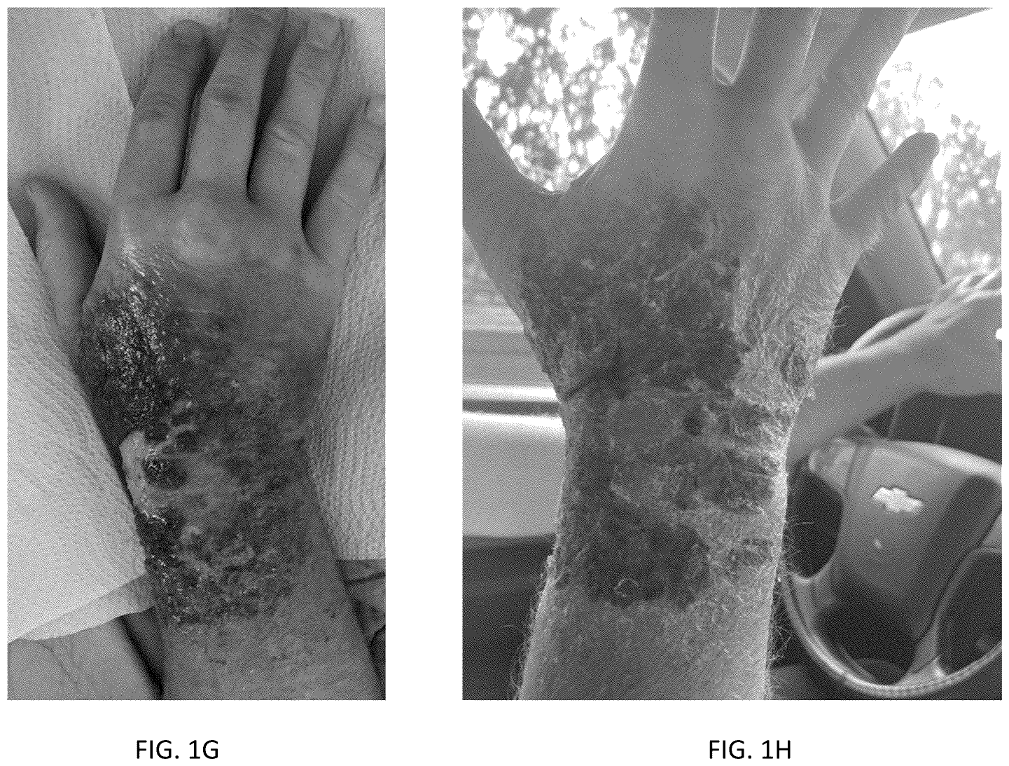

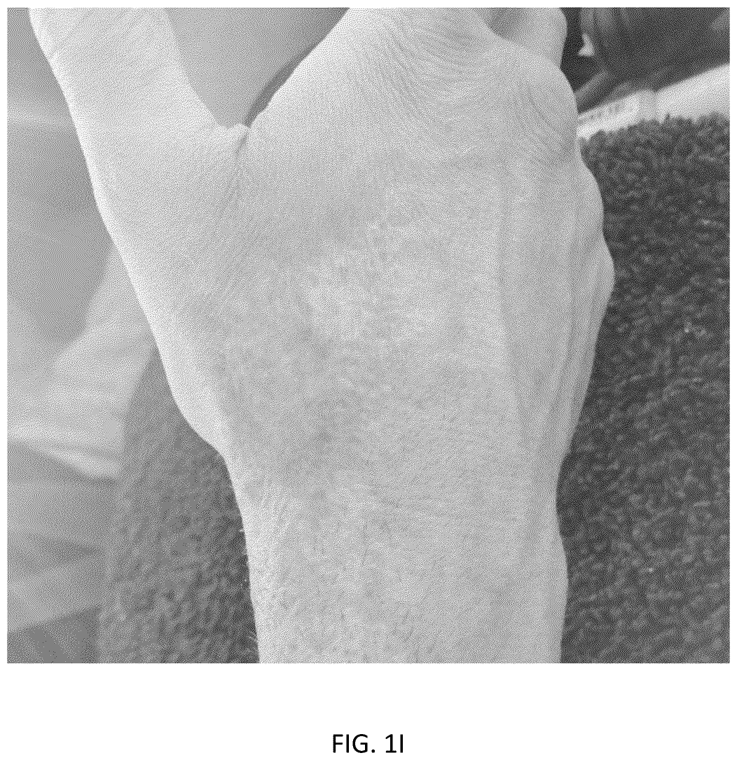

This example shows the treatment of an antibiotic-resistant infection. Several types of antibiotic drugs, including intravenous antibiotics, were used to treat the infection but were not effective. The infection area after the antibiotic treatments is shown in FIGS. 1A and 1B.

Then an exemplary salve comprising red clover, bloodroot, galangal, and sheep sorrel according to the present disclosure was applied on the infection area and left on without any cover for a time period between one and two minutes. The salve penetrated the infection and formed white, yellow, grey eschars (see FIG. 1C). FIG. 1D shows the area a few hours after the salve application. The infection stopped spreading and was beginning to dry up. FIG. 1E shows the area one day following the salve application. The infection was no longer active, and the skin was drying up and turning to scab. FIG. 1F shows the area 2 days after the salve application. The skin was dried and scabbed, and was healing. FIG. 1G shows the area 5 days after the salve application. The infection had dried and scabbed, and was slowly peeling as skin healing underneath. FIG. 1H shows the area 10 days after the salve application. The skin was recovering and getting back to normal. The infection was gone, and skin with normal functioning and overall health was restored.

In this case, conventional antibiotic medicines failed and had an extremely negative impact on the body and only exacerbated the infection. This case shows the exemplary salve according to the present disclosure cured the infection within a one- to two-minute application. FIG. 1I shows the area a few months after the treatment. The skin was like new, only with the scar.

### Example 3

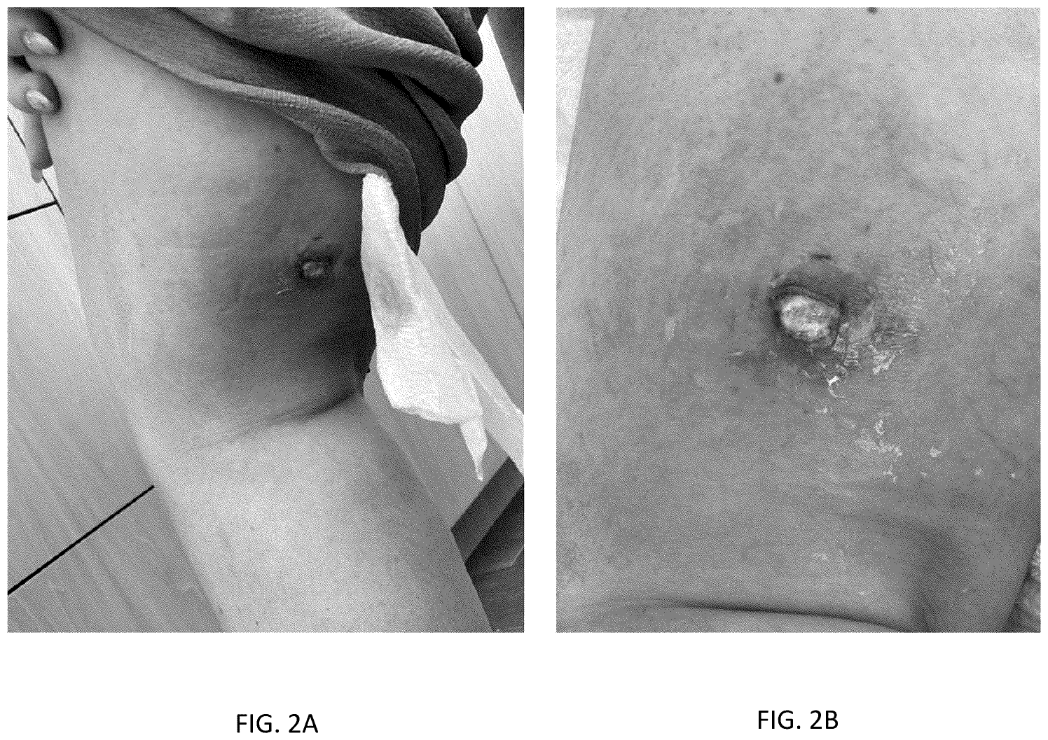

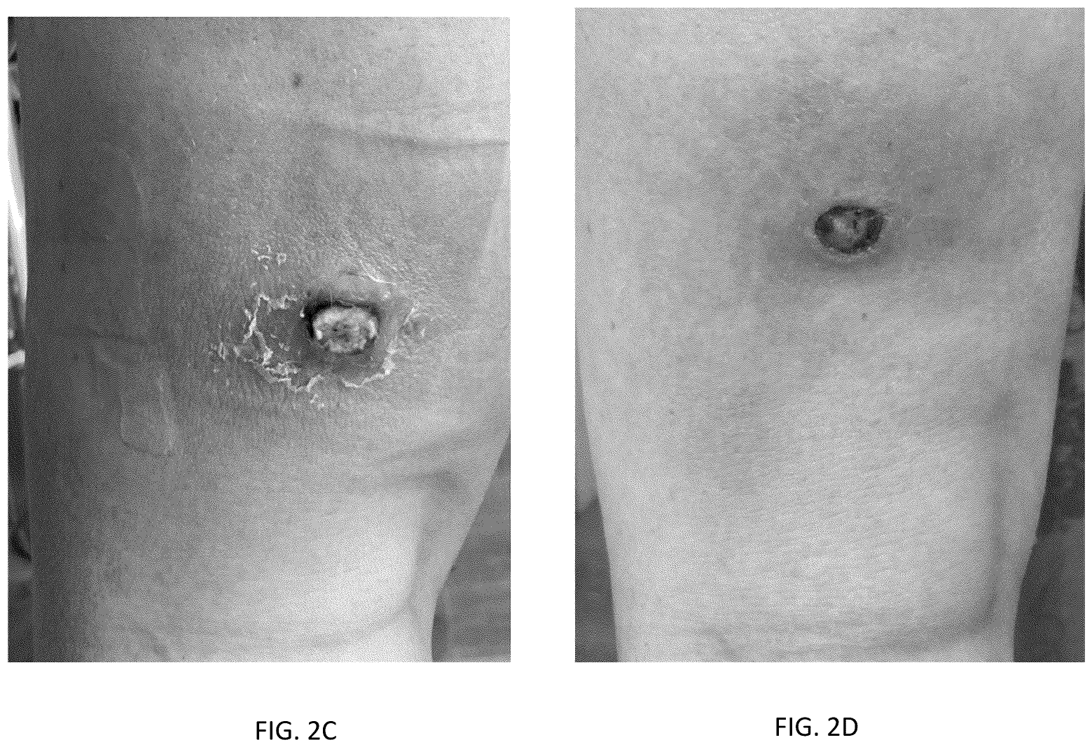

This example shows the treatment of an infected spider bite. FIG. 2A shows the infected area. The patient's leg was very swollen and red. Doctors had been draining and applying topical antibiotic creams for 5 days already, and the patient was taking prescribed antibiotics, both of which had not helped, and the infection was worsening.

An exemplary salve comprising red clover, bloodroot, galangal, and sheep sorrel according to the present disclosure was applied on the area without any cover for 1 minute. FIG. 2B shows the area immediate after the salve application. FIG. 2C shows the area 24 hours after the salve was applied. The salve formed an eschar in the area. The infection and swelling had receded. FIG. 2D shows the area 5 days after the salve application. The infection was gone, eschar had turned to scab, which was healing naturally and easily.

This case shows the different effects of 5-day treatment with conventional antibiotics and 5-day treatment with the exemplary salve according to the present disclosure.

### Example 4

This example demonstrates treating an infection resulting from a spider bite or oral thrush in a patient's mouth. An exemplary salve comprising red clover, bloodroot, galangal, and sheep sorrel according to the present disclosure was applied on the area without cover for 15 to 20 seconds, and washed off. The area took about a week to heal completely. The infection and related symptoms stopped during the process and the infection vanished successfully. When applying the salve, caution was taken to avoid the teeth.

Various modifications and variations of the described methods and compositions of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of treating a subject with a topical condition, comprising applying to a topical surface of the subject a composition comprising red clover, bloodroot, galangal, and sheep sorrel, wherein the topical condition is an infection, animal bite, or dermatitis, and wherein the composition is kept on the topical surface for no more than 12 hours without a cover.

2. The method of claim 1, wherein the topical surface is skin.

3. The method of claim 1, wherein the topical surface is oral mucosa.

4. The method of claim 1, wherein the composition is kept on the topical surface for no more than 1 hour.

5. The method of claim 1, wherein the composition is kept on the topical surface for no more than 5 minutes.

6. The method of claim 1, wherein the composition is kept on the topical surface for no more than 2 minutes.

7. The method of claim 1, wherein the composition is kept on the topical surface for no more than 1 minute.

8. The method of claim 1, wherein the topical condition is an infection.

9. The method of claim 8, wherein the infection is a bacterial or fungal infection.

10. The method of claim 8, wherein the infection is cellulitis, staph, MRSA, or jock itch.

11. The method of claim 1, wherein the topical condition is an animal bite.

12. The method of claim 11, wherein the topical condition is an insect bite or spider bite.

13. The method of claim 1, wherein the topical condition is dermatitis.

14. The method of claim 13, wherein the dermatitis is eczema, psoriasis, or rash.

15. The method of claim 1, wherein the composition is a paste, ointment, or salve.

16. The method of claim 1, comprising applying the composition to the topical surface more than one time.

17. A method of treating a subject with a topical condition, comprising applying to a topical surface of the subject a composition comprising an extract from bloodroot, wherein the topical condition is an infection, animal bite, or dermatitis.

18. The method of claim 17, wherein the extract from bloodroot is bloodroot juice.

19. The method of claim 17, wherein the composition is kept on the topical surface for eight to 24 hours without a cover.
